(19) United States
(12) Patent Application Publication
(10) Pub. No.: US 2007/0003480 A1
(43) Pub. Date: Jan. 4, 2007

(54) Liposomal composition comprising haptotactic peptides

(75) Inventors: Gerard Marx, Jerusalem (IL); Raphael Gorodetsky, Jerusalem (IL)

Correspondence Address: HESLIN ROTHENBERG FARLEY & MESITI PC, 5 COLUMBIA CIRCLE, ALBANY, NY, 12203, US

(73) Assignee: Hapto Biotech, Inc., Garden City, NJ (US)

(21) Appl. No.: 10/533,826

(22) PCT Filed: Nov. 3, 2003
(86) PCT No.: PCT/IL03/00911

§ 371 Date: Apr. 6, 2006

(30) Foreign Application Priority Data
Nov. 3, 2002 (IL) 152609

(51) Int. Cl. A61K 51/00; A61K 39/395; A61K 49/10; A61K 9/127

(52) U.S. Cl. 424/1.21; 424/450; 424/9.34; 424/133.1

(57) ABSTRACT

The present invention relates to liposomal composition comprising peptides characterized in that they elicit cell attachment (haptotactic) responses and are internalized by cells; more particularly the compositions comprise cell attachment peptides derived from or homologous to specific portions of the carboxy termini of fibrinogen chains, designated herein as Haptides, capable of enhancing the uptake of liposomes by cells. The present invention further relates to pharmaceutical and cosmetic compositions comprising haptotactic-peptide liposomal compositions and uses of same.

### FIELD OF THE INVENTION

The present invention relates to liposomal compositions comprising peptides characterized in that they elicit cell attachment (haptotactic) responses and are internalized by cells; more particularly the compositions comprise cell attachment peptides derived from or homologous to specific portions of the carboxy termini of fibrinogen chains, designated herein as Haptides, capable of enhancing the uptake of liposomes by cells. The present invention further relates to pharmaceutical and cosmetic compositions comprising haptotactic-peptide liposomal compositions and uses of same.

### BACKGROUND OF THE INVENTION

Fibrinogen is the plasma protein responsible for blood clot formation. Normal fibrinogen (MW 340 kDa) is a complex hexamer composed of two sets of three non-identical chains (α, β and γ) linked by multiple disulfide bonds. A larger variant of fibrinogen (MW 420 kDa), with an extended a chain, has also been described (Grieninger G. 2001. Ann NY Acad Sci 936:44-64).

Fibrinogen is not immunogenic within the same species, as attested by the use of pooled fibrin glue for clinical applications. Besides its hemostatic activity, it has been previously demonstrated that fibrin(ogen) elicits cell attachment (haptotactic) and migratory (chemotactic) responses with different cell types including mouse and human fibroblasts (MF and HF), bovine aortic endothelial cells (BAEC) and smooth muscle cells (SMC) (Gorodetsky R. et al. 1999. J Invest Dermatol 112:866-872; Gorodetsky R. et al. 1998. J Lab Clin Med 131:269-280; Gurevich et al. 2002. Tissue Engineering, Vol. 8).

The inventors of the present invention have previously disclosed (WO99/61041; WO01/53324) synthetic peptides having sequences derived from or homologous to the conserved carboxy-terminus of fibrinogens, particularly to the carboxy-terminus of the β- and γ chains that appear also in a few other proteins, such as tenascins, microfibril associated glycoproteins and angiopoietins. When bound to matrices, the disclosed 17-21 mer homologous peptides, as well as some shorter 8-10 mer sequences within the peptides elicited cell attachment (haptotactic) responses from different cell types, including normal fibroblasts, endothelial cells and smooth muscle cells. These peptides were therefore designated “Haptides”. None of the Haptides altered the rates of cell proliferation of the different cell types tested.

Fibrinogen exhibits substantial hydrophobic character, as evidenced by its ability to bind to various lipids and fatty acids (Cunningham M. T. et al. 1999. Thromb Res 95: 325-34; Nygren H. et al. 1992. J Biomed Mater Res 26: 77-91; Retzinger G. S. et al. 1998. Arterioscler Thromb Vasc Biol 18: 1948-57). For example, fibrinogen could be adsorbed onto hydrophobic surfaces coated with cholesteryl oleate, cholesterol, or lecithin. In that context, it has been proposed that the affinity of fibrinogen for hydrophobic, atheromatous lipid surfaces, particularly those rich in cholesteryl esters, may predispose these surfaces to thrombosis. The hydrophobic nature of fibrinogen, leading to its ability to interact with and entrap liposomes, was used in developing fibrin-liposome compositions as a slow, topical drug delivery system (U.S. Pat. No. 5,607,694 to Marx, one of the present inventors).

The therapeutic, diagnostic or cosmetic benefit of many compounds is limited by low uptake of the compound by the target cells or by intracellular breakdown of the compound after uptake. For many compounds, permeation across the cell membrane may allow relatively efficient uptake by the cell. However, for a variety of larger and/or charged compounds, such as proteins, nucleic acids and many organic compounds, passive uptake by penetration across the cell membrane is limited. This phenomenon limits the use of many efficient medications, and/or requires the use of high doses that may cause undesired systemic drug toxicity.

Several methods for improving uptake of such compounds have been proposed. One common method is based on modifying the compound by forming a reversible complex with a carrier group that improves penetration (diffusion) into the cell wherein the compound is effective. Another approach is to use liposomes designed to fuse with the surface membrane of a target cell to release the particle contents into the cytoplasmic compartment of the cell.

Liposomes are defined as a structure consisting of one or more concentric lipid bilayers separated by water or aqueous buffer compartments. These hollow structures, which have an internal aqueous compartment, can be prepared with diameters ranging from 20 nm to 10 μm. They are classified according to their final size and preparation method as SUV, small unilamellar vesicles (0.5-50 nm); LUV, Large unilamellar vesicles (100 nm); REV, reverse phase evaporation vesicles (0.5 μm); and MLV, multilamellar large vesicles (2-10 μm). Depending on their composition and storage conditions, liposomes exhibit varying degrees of stability. The core micro-reservoirs of liposomes and the space between the bilayers can contain a variety of water-soluble materials (Davis S. S. & Walker I. M. 1987. Methods in Enzymology 149: 51-64; Gregorius G. (Ed) 1991. Liposomes Technology Vols I, II, III. CRC Press, Boca Raton, Fla.; Shafer-Korting M. et al. 1989 J Am Acad Dermatol 21: 1271-1275). Liposomes can also serve as carriers for lipophilic molecules intercalated into the lipid bilayer.

Other forms of artificially created vesicles whose outer wall contains molecules that enable their fusion with a cell membrane include inactivated and reconstituted virus particles and specific types of emulsions.

Reconstituted virus particles and artificial virus-like particles are mainly used in gene therapy where the object is to introduce large nucleic acid strands into the cells. However, although viral vectors have certain advantages, including high levels of transfection, or efficient and stable integration of foreign DNA into a wide range of host genomes, they suffer from several problems including immunogenicity, toxicity, difficulty of large-scale production, size limit of the exogenous DNA, random integration into the host genome, and the risks of inducing tumorigenic mutations and/or generating active viral particles through recombination. These problems, especially the safety concerns, limit the use of virus-particles for facilitating uptake of impermeable substances into cells.

Emulsions are defined as heterogeneous system in which two immiscible liquids are dispersed one in the other. Such dispersions (oil in water or water in oil) are stabilized by emulsifiers that coat the droplet to prevent droplet coalescence. Emulsions are usually used as a means of administering aqueous-insoluble drugs by dissolution of the drug within the oil phase. The droplets size in such emulsions for medical applications is usually at the sub-micron range (International Application No. WO 96/33697; U.S. Pat. Nos. 5,496,811; 5,514,670; 5,961,970; 5,993,846; 6,113,921).

Emulsomes are solid fat nano-emulsions that are intermediate between liposomes and oil-in water emulsions. The nanoparticles contain a hydrophobic core surrounded and stabilized by one or more layers of phospholipid layers. This structure enables loading of hydrophobic molecules in the internal solid lipid core and hydrophilic molecules in the aqueous compartments of surrounding phospholipid layers (U.S. Pat. No. 5,576,016).

There is an ongoing effort to improve the efficiency of liposomes as a delivery system for therapeutic agents. For example, International Application No. WO 02/076491 discloses the use of small matrix metalloproteinase inhibitors in improving targeting of liposomes to cancer cells, and in enhancing the uptake of the liposomes by such cells.

Another approach has been based on the promotion of cellular internalization of liposomes through receptor-mediated endocytosis. This approach may be combined with a method for facilitating the cytoplasmic release of the desired compound from endosomes by the association of endosome-disrupting agents to the liposome, thus preventing the lysosomal degradation of the compound. International Application No. WO 02/076428 discloses liposomes composed of a pH sensitive lipid that also include a targeting ligand to direct the liposomes to a target cell. Administration of the liposomes results in cellular internalization and destabilization of the liposome for intracellular delivery of the entrapped agent.

However, the use of receptor-specific ligands as a targeting mechanism for liposomes serving as delivery vehicles presents several problems, both in vitro and in vivo. Receptor-specific ligands are relatively rare molecules and incur considerable expense in isolating and collecting an adequate supply. In addition, receptor-ligands are usually potent effectors of many biological responses, often linked to turning “on” and “off” of cell proliferation. Therefore, the use of such receptor-directed (specific) ligands with liposomes poses a potentially serious threat of adverse or unwanted side effects, mainly increased cell proliferation or apoptosis.

Thus, there is a recognized need for, and it would be highly advantageous to have an improved system for the delivery of lipsomes and other fusogenic vesicles into cells without modulating cell proliferative or apoptotic responses; such improvement would be highly beneficial for therapeutic, diagnostic and cosmetic uses.

### SUMMARY OF THE INVENTION

The present invention relates to liposomal compositions comprising peptides characterized in that they elicit cell attachment (haptotactic) activity and are internalized by cells, providing improved intracellular uptake of liposomes, wherein the liposomes of the compositions may comprise compounds having diagnostic, therapeutic or cosmetic activity.

The term “haptotactic-peptide liposomal compositions” as used herein refers to compositions comprising liposomes and at least one type of Haptide. The term “Haptide” refers to a peptide derived from, or homologous to haptotactic peptides present within the carboxy-termini of the fibrinogen chains, and is characterized in that it elicit cell attachment (haptotactic) response, and, when in a free form, is readily taken up by various cell types. The terms “haptotatic peptide” and “Haptide” are used herein interchangeably.

The present invention provides methods for enhancing liposome uptake by a cell, using the haptotactic-peptide liposomal compositions. The present invention further provides pharmaceutical and cosmetic compositions which comprise a haptotactic-peptide liposomal composition.

According to one aspect, the present invention provides liposomal compositions comprising Haptides. The liposomes of the present invention may be of variable types as is known in the art, comprising at least one hydrophilic and at least one hydrophobic compartment.

According to one embodiment, the present invention provides haptotactic-peptide liposomal compositions comprising at least one type of Haptide and one type of liposome.

According to one embodiment of the present invention the haptotactic-peptide liposomal compositions comprise at least one type of Haptide having an amino acid sequence which is at least 60% homologous to a C-terminus sequence of fibrinogen β, αE and γ chains, preferably 70%, more preferably 80% most preferably 90% or more homologous to the C-termini of the fibrinogen chains, and fragments, variants, analogs and pepitidomimetic thereof which retain the haptotactic activity.

According to one embodiment, the present invention provides haptotactic peptides comprising amino acid sequences of 6-40 amino acids residues, preferably 7-30 residues, more preferably 8-25 amino acids residues.

According to one currently preferred embodiment the haptotactic-peptide liposomal compositions comprise Haptides selected from the group consisting of 17-21 mer peptides having the following amino acids sequences:

| | |
|---|---|
| KGSWYSMRKMSMKIRPFFPQQ; | (peptide Cβ, |
| | SEQ ID NO:1) |
| RGADYSLRAVRMKIRPLTVTQ; | (peptide CαE, |
| | SEQ ID NO:2) |
| KTRWYSMKKTTMKIIPFNRL; | (peptide preCγ, |
| | SEQ ID NO:3) |
| KGPSYSLRSTTMMIRPLDF; | (peptide-C-ang1, |
| | SEQ ID NO:4) |
| KGSGYSLKATTMMIRPADF; | (peptide-C-ang2, |
| | SEQ ID NO:5) |
| KGFEFSVPFTEMKLRPNFR, | (peptide-C-tenX, |
| | SEQ ID NO:6) |
| and | |
| KGFYYSLKRPEMKIRRA. | (peptide-C-mfap, |
| | SEQ ID NO:7) |

According to additional currently preferred embodiments the haptotactic peptides are shorter sequences comprising 8-10 mer peptides having the following amino acid sequences:

| | |
|---|---|
| KGSWYSMR; | (peptide-Cβ₈, SEQ ID NO:8) |
| KGSWYSMRKM; | (peptide-Cβ₁₀, SEQ ID NO:9) |
| KTRWYSMKKT; | (peptide-PreCγ₁₀, SEQ ID NO:10) |
| KGPSYSLR | (peptide-C-ang1₈, (SEQ ID NO:11) |
| and | |
| KGFYYSLKRP. | (peptide-C-mfap₁₀, (SEQ ID NO:12) |

According to yet further currently preferred embodiment the haptotactic peptides are synthesized according to the cell attachment and internalizing consensus sequences selected from the following amino acids sequences:

| | |
|---|---|
| KGXXYSMRKXXMKIRP | (SEQ ID NO:13) |
| and | |
| KGXXYSMRK, | (SEQ ID NO:14) |

wherein

X denotes a non-charged amino acid, or may be absent thereby forming a direct bond.

It should be noted that conservative replacements of the amino acid residues of these sequences, as well as other modifications such as extensions and truncations which retain the haptotactic activity are also encompassed within the scope of the present invention, as is well known in the art.

According to one currently most preferred embodiment, the haptotactic peptide selected for the haptide-liposomal composition is Cβ or preCγ.

According to one embodiment the compositions of the present invention may comprise fusogenic vesicles other than liposomes. Fusogenic vesicles are defined as artificially created vesicles whose outer walls contain molecules that enable their fusion with a cell membrane. Common examples of fusogenic vesicles other than liposomes are inactivated and reconstituted virus particles and specific types of emulsions.

The liposomes of the compositions of the present invention may be of any suitable biocompatible variety, comprising at least one hydrophilic and at least one hydrophobic compartment, wherein the hydrophobic compartment comprises at least one lipid bilayer.

According to one embodiment the liposomes of the present invention comprise vesicle-forming lipids, each lipid comprising a hydrophilic “head” group and a hydrophobic “tail” group. The basic lamella may be a monolayer, such as in emulsions, or a bilayer formed by “tail to tail” interactions of the lipids, such as in liposomes. The vesicles may be unilamellar or multilamellar. The liposomes may further comprise stabilizers and surfactants.

According to one embodiment the liposomes comprise at least one of the following substances: phospholipids of natural or synthetic origin; phospholipids combined with glycerides; phospholipids combined with polyethylene glycol (PEG); phosphoaminolipids; cerebroglucosides and gangliosides; optionally further comprising natural or synthetic cholesterol and hydrogenated lecithin as non-limiting examples.

The liposomes of the present invention may further comprise biologically active compounds. Molecules advantageously included within the liposomes include, but are not limited to molecules having diagnostic, therapeutic or cosmetic activity. Such molecules are exemplified by polynucleotides, proteins, peptides, polysaccharides, hormones, drugs, vitamins, steroids, fluorescent dyes, radioactive markers and the like.

According to another aspect, the present invention provides a method for enhancing liposomes uptake into cells, in vitro or in vivo.

According to one embodiment the present invention provides a method for enhancing uptake of liposomes into cells comprising:

providing a haptotactic-peptide liposomal composition; and

contacting cells with the haptotactic-peptide liposomal composition;

wherein liposomal uptake by the cells is enhanced at least two fold compared to the uptake of said liposomes without the haptotactic peptide.

According to one embodiment, the haptotactic peptide-liposomal composition is produced ab initio with at least one Haptide. According to another embodiment, the composition is produced extemporaneously using preformed vesicles combined with at least one type of Haptide.

According to one embodiment of the present invention, the method of producing a haptotactic-peptide liposomal composition comprises the step of dispersing liposomal components with a solution of at least one type of haptotactic peptide in an aqueous buffer.

According to one embodiment of the present invention, the cells to which the liposomes are directed are selected from the group consisting of mammalian cells including leukocytes, and cells from mesenchymal origin including astrocytes, chondrocytes, dendritic cells, endothelial cells, fibroblasts, glial cells, neurons, kidney cells, liver cells, melanocytes, mesenchymal cells, myofibroblasts, monocytes, parenchymal cells, pancreatic cells, smooth muscle cells and thyroid cells as well as malignant and transformed cells of any origin.

According to yet another aspect the present invention provides a method for enhancing the intracellular uptake of biologically active molecules that would otherwise have low permeability through the cell membrane.

According to one embodiment the present invention provides a method for using haptotactic-peptide liposomal compositions for enhanced intracellular uptake of biologically active molecules characterized by low-permeability through the cell membrane, the method comprising:

providing a haptotactic-peptide liposomal composition wherein the liposomes further comprise biologically active molecules characterized by low-permeability through the cell membrane; and

contacting cells with the haptotactic-peptide liposomal composition;

wherein the molecules uptake is enhanced at least two fold compared to the uptake of said molecules detached from said haptotactic-peptide liposomal composition.

According to one embodiment, the haptotactic peptide-liposomal composition which further comprises molecules characterized by low-permeability through the cell membrane is produced ab initio with at least one type of Haptide and at least one type of the low-permeability molecule. According to another embodiment, the composition is produced extemporaneously using preformed vesicles comprising at least one type of low-permeability molecule combined with at least one type of Haptide.

According to one embodiment, the biologically active molecules are at least partially lipid soluble and are present in the hydrophobic lipid bilayer.

According to another embodiment, the biologically active molecules are water-soluble and are present in the aqueous compartment of the liposomes.

According to one embodiment, the biologically active molecules within the liposomes are selected from the group consisting of polynucleotides, proteins, peptides, polysaccharides, hormones, drugs, steroids, fluorescent dyes and radioactive markers.

According to a further aspect the present invention provides pharmaceutical and cosmetic composition comprising haptotactic-peptide liposomal compositions wherein the liposomes further comprise an active ingredient having diagnostic, therapeutic or cosmetic activity, respectively.

According to one embodiment the present invention provides a pharmaceutical composition comprising haptotactic peptide-liposomal composition, wherein the peptides are haptotactic peptides characterized in that they elicit cell attachment activity and are internalized by cells and the liposomes further comprise an active ingredient having a diagnostic or therapeutic activity, further comprising a pharmaceutically acceptable diluent or carrier.

According to one preferred embodiment, the active ingredient within the liposomes of the pharmaceutical composition is selected from the group consisting of a cytotoxic compound, a cytostatic compound, an antisense compound, an anti-viral agent, a specific antibody and an imaging agent.

According to another embodiment, the present invention provides a cosmetic composition comprising haptotactic peptide-liposomal composition, wherein the peptides are haptotactic peptides characterized in that they elicit cell attachment activity and are internalized by cells, and the liposomes further comprise an active ingredient having a cosmetic beneficial effect, further comprising a cosmetically acceptable diluent or carrier.

According to yet another aspect the present invention relates to methods for treating a subject in need thereof with the pharmaceutical or cosmetic compositions of the present invention.

According to one embodiment the present invention provides a method for enhancing the delivery of a pharmaceutical agent into cells comprising the step of administering to a subject in need thereof a therapeutically effective amount of haptotactic peptide-liposomal pharmaceutical composition wherein the liposomes of the composition further comprise the pharmaceutically effective agent.

According to another embodiment the present invention provides a method for enhancing the delivery of a diagnostic agent into cells comprising the step of administering to a subject in need thereof a diagnostically effective amount of a haptotactic peptide-liposomal pharmaceutical composition, wherein the liposomes of the composition further comprise the diagnostically effective agent.

According to yet another embodiment the present invention provides a method for enhancing the delivery of a cosmetically effective liposomes into cells comprising the step of administering to a subject in need thereof a haptotactic peptide-liposomal cosmetic composition wherein the liposomes of the composition have a cosmetic beneficial effect. These liposomes may further comprise a cosmetically effective agent.

According to further embodiment the present invention provides the use of a haptotactic peptide-liposomal pharmaceutical composition wherein the liposomes of the composition further comprise a pharmaceutically effective agent for enhancing the delivery of the pharmaceutically effective agent into cells.

According to yet further embodiment the present invention provides the use of a haptotactic peptide-liposomal pharmaceutical composition wherein the liposomes of the composition further comprise a diagnostically effective agent for enhancing the delivery of the diagnostically effective agent into cells.

According to another embodiment the present invention provides the use of a haptotactic peptide-liposomal pharmaceutical composition wherein the liposomes of the composition have a cosmetically beneficial effect for enhancing the delivery of the cosmetically effective liposomes into cells. These liposomes may further comprise a cosmetically effective agent.

The present invention is explained in greater detail in the description, Figures and claims below.

### BRIEF DESCRIPTION OF THE DRAWINGS

The invention is herein described by way of example only, with reference to the accompanying drawings wherein:

FIG. 1 shows the uptake of free fluorescein isothiocyanate (FITC)-labeled haptotactic peptides (Haptides) by human fibroblasts measured by confocal fluorescence microscopy

FIG. 2 shows, by confocal fluorescence microscopy, the association of ^{FITC}fibrinogen (A) and ^{FITC}Haptides (B-Cβ, C-preCγ, D-CαE) with liposomes.

FIG. 3 shows the average particle size of the haptotactic peptide liposomal composition compared to the size of Haptide aggregates and liposomes, measured by dynamic light scattering.

FIG. 4 shows haptotactic-peptide augmented uptake of rhodamine-loaded liposomes by human fibroblasts, measured by confocal microscopy. FIG. 4A: no Haptides. FIG. 4B: Haptide Cα. FIG. 4C: Haptide CαE. FIG. 4D: Haptide preCγ. FIG. 4E: Haptide Cβ.

FIG. 5 shows haptotactic-peptide-augmented uptake of Doxil (Doxorubicin loaded) liposomes by bovine aortic endothelial cells, measured by confocal fluorescence microscopy. FIG. 5A: no Haptides. FIG. 5B: Haptide Cα. FIG. 5C: Haptide Cβ. FIG. 5D: Haptide PreCγ. FIG. 5E: Haptide CαE.

FIG. 6 shows uptake of Doxorubicin-loaded liposomes alone compared to the uptake of Haptide-Doxorubicin-loaded liposome composition. Liposome uptake was directly correlated to the Haptide (Cβ) concentration in the Haptide-Doxorubicin-loaded liposomes composition.

FIG. 7 shows uptake of Haptide-Doxil liposomal composition by human fibroblast compared to the uptake of Doxil liposome alone, as measured by the cytotoxic activity (influence on cell survival) of Doxil.

FIG. 8 schematically depicts the principle underlying liposomes uptake mediated by Haptides.

### DETAILED DESCRIPTION OF THE INVENTION

The present invention relates to liposomal compositions comprising haptotactic peptides that are readily taken by cells and to uses of same for delivery of liposomes comprising biologically active molecules through a cell membrane, in vitro or in vivo. The liposomal compositions are designated herein as haptotactic-peptide liposomal compositions.

As used herein, haptotactic peptides, or Haptides, are peptides derived from, or homologous to haptotactic peptides present within the carboxy-termini of the fibrinogen chains, and are characterized in that they elicit cell attachment responses from cultured cells. As defined herein, cell attachment refers to any kind of cell-Haptide interaction, including covalent or non-covalent cell binding, cell adhering, and Haptide-cell complex formation.

Haptides are also characterized in that they are readily taken up by different cell types, and can therefore induce augmented uptake of different substances into the cells.

As used herein the term “peptide” indicates a sequence of amino acids linked by peptide bonds. The peptide analogs of this invention comprise a sequence of amino acids of 6 to 40 amino acid residues, preferably 7 to 30 residues, more preferably 8-25 amino acids residues, each residue being characterized by having an amino and a carboxy terminus.

The haptotactic-peptide liposomal compositions of the present invention comprise haptotactic peptides and liposomes in a ratio of at least one haptotactic peptide molecule per liposome.

The haptotactic peptides (Haptides) of the present invention are peptides derived from or homologous to the C-termini of a fibrinogen chain, characterized in that they mimic the parent property of cell adhesive effect. The active peptide may be a fragment of a natural protein, a fragment of a recombinant protein, or, preferably, a synthetic peptide.

According to a preferred embodiment, the Haptides of the present invention have an amino acid sequence which is at least 60% homologues, preferably 70%, more preferably 80% and most preferably 90% or more homologous to the C-termini of the fibrinogen αE, β or γ chain, and fragments, variants, analogs and pepitidomimetic thereof which retain the haptotactic activity.

As defined herein, amino acids sequence homology is typically measured using sequence analysis software (for example, Sequence Analysis Software Package of the Genetics Computer Group (University of Wisconsin Biotechnology Center, 1710 University Avenue, Madison, Wis. 53705), BLAST, or PILEUP/PRETTYBOX programs). Such software matches sequences by assigning degrees of homology to various substitutions, deletions, and other modifications.

According to one currently preferred embodiment, the haptotactic peptides of the present invention are selected from the group consisting of 17-21 mer peptides having the following amino acid sequences:

| | |
|---|---|
| KGSWYSMRKMSMKIRPFFPQQ; | (peptide Cβ, |
| | SEQ ID NO:1) |
| RGADYSLRAVRMKIRPLTVTQ; | (peptide CαE, |
| | SEQ ID NO:2) |
| KTRWYSMKKTTMKIIPFNRL; | (peptide preCγ, |
| | SEQ ID NO:3) |
| KGPSYSLRSTTMMIRPLDF; | (peptide-C-ang1, |
| | SEQ ID NO:4) |
| KGSGYSLKATTMMIRPADF; | (peptide-C-ang2, |
| | SEQ ID NO:5) |
| KGFEFSVPFTEMKLRPNFR, | (peptide-C-tenX, |
| | SEQ ID NO:6) |
| and | |
| KGFYYSLKRPEMKIRRA, | (peptide-C-mfap, |
| | SEQ ID NO:7) |

According to additional currently preferred embodiments the haptotactic peptides are shorter sequences comprising 8-10 mer peptides having the following amino acid sequences:

| | |
|---|---|
| KGSWYSMR; | (peptide-Cβ₈, |
| | SEQ ID NO:8) |
| KGSWYSMRKM; | (peptide-Cβ₁₀, |
| | SEQ ID NO:9) |
| KTRWYSMKKT; | (peptide-PreCγ₁₀, |
| | SEQ ID NO:10) |
| KGPSYSLR | (peptide-C-ang₈, |
| | (SEQ ID NO:11) |
| and | |
| KGFYYSLKRP. | (peptide-C-mfap₁₀, |
| | (SEQ ID NO:12) |

According to yet further currently preferred embodiment the haptotactic peptides are synthesized according to the cell attachment and internalizing consensus sequences:

| | |
|---|---|
| KGXXYSMRKXXMKIRP | (SEQ ID NO:13) |
| and | |
| KGXXYSMRK, | (SEQ ID NO:14) |

wherein X denotes a non-charged amino acid, or may be absent thereby forming a direct bond.

“Amino acid sequence”, as used herein, refers to an oligopeptide, peptide, polypeptide, or protein sequence, and fragment thereof, and to naturally occurring or synthetic molecules, which retain the haptotactic activity according to the present invention.

The term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite haptotactic activity as specified herein.

As used herein, a peptide derivative refers to a peptide having an amino acid sequence that comprises the amino acid sequence of the peptide of the invention. Thus, the peptides of the present invention can be subject to various changes, substitutions, insertions, and deletions where such changes do not destroy the haptotactic activity of the peptide.

According to one currently most preferred embodiment, the haptotactic peptide selected for the haptotactic-peptide liposomal compositions is Cβ or preCγ.

The liposomes comprising at least one Haptide according to the present invention may be of any suitable biocompatible variety, comprising at least one hydrophilic and at least one hydrophobic compartment, wherein the hydrophobic compartment comprises at least one lipid bilayer.

The liposomes of the present invention comprise vesicle-forming lipids, each lipid composed of hydrophilic “head” group and a hydrophobic “tail” group. It is to be emphasized that although the preferred embodiments of the present invention refer to haptotactic-peptide liposomal compositions, other fusogenic vesicles can also be used. Fusogenic vesicles are defined as artificially created vesicles whose outer wall contains molecules that enable their fusion with a cell membrane. Common examples of fusogenic vesicles are inactivated and reconstituted virus particles, specific types of emulsions and liposomes.

The vesicle lamella may be a monolayer, such as in emulsions, or a bilayer, formed “tail to tail” such as in liposomes. The vesicle may be uni-or multi-lamellar. The liposomes may further comprise stabilizers and surfactants.

Materials and methods for forming liposomes are well known to those skilled in the art and will only briefly described herein. Upon dispersion in appropriate medium, a wide variety of phospholipids swell, hydrate and form multilamellar concentric bilayer vesicles with layers of aqueous media separating the lipid bilayers. These systems, first described by Bangham et al. (1965. J Mol Biol 13:238-252) are referred to as multilamellar lipid vesicle (“MLVs”) and have diameters with the range of 10 nm to 100 μm. In general, lipids or lipophilic substances are dissolved in an organic solvent. When the solvent is removed, such under vacuum by rotary evaporation, the lipid residue form a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reducing their size by other forms of mechanical shearing. There are also techniques by which MLVs can be reduced both in size and in number of lamellae, for example, by pressurized extrusion (Barenholz et al. 1979. FEBS Lett 99:210-214).

Liposomes can also take the form of unilamellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicle. In Papahadjopoulos et al. (1968. Biochim et Biophys Acta 135:624-638), sonication of an aqueous dispersion of phospholipids produces small ULVs having a lipid bilayer surrounding an aqueous solution. U.S. Pat. No. 4,089,801 to Schneider describes the formation of liposomes precursors by ultrasonication, followed by the addition of an aqueous medium containing amphiphilic compounds and centrifugation to form a biomolecular lipid layer system.

Small ULVs can also be prepared by the ethanol injection techniques described by Batzri et al. (1973. Biochim et Biophys Acta 298:1015-1019) and the ether injection technique of Deamer et al. (1976. Biochim et Biophys Acta 443:629-634). These methods involve the rapid injection of an organic solution of lipids into a buffer solution, which results in the rapid formation of unilamellar liposomes. Another technique for making ULVs is taught by Weder et al. (1984. In “Liposome Technology” ed. Gregoriadis G. CRC Press Inc. Boca Raton Fla. Vol I Chapter 7 pp. 79-107). This detergent removal method involves solubilizing the lipids and additives with detergents by agitation or sonication to produce the desired vesicles.

In addition to the MLVs and ULVs, liposomes can also be multivesicular. As described in Kim et al. (1983. Biochim et Biophys Acta 728:339-348), these multivesicular liposomes are spherical and contain internal granular structures. The outer membrane surface is a lipid bilayer and the internal region contains small compartments separated by bilayer septum. Still yet another type of liposomes is oligolamellar vesicles (“OLVs”), which have a large centre compartment surrounded by several peripheral lipid layers. These vesicles, having a diameter of 2-15 μm, are described in Callo et al. (1985. Cryobiology 22(3):251-267).

U.S. Pat. Nos. 4,485,054 and 4,761,288 to Mezei et al. also describe methods of preparing lipid vesicles. U.S. Pat. No. 5,653,996 to Hsu describes a method of preparing liposomes utilizing aerosolization and U.S. Pat. No. 5,013,497 to Yiournas et al. describes a method of preparing liposome utilizing a high velocity shear-mixing chamber. Methods are also described that use specific starting materials to produce ULVs (for example, U.S. Pat. No. 4,853,228) or OLVs (for example U.S. Pat. Nos. 5,474,848 and 5,628,936).

A comprehensive review of lipid vesicles and methods for their preparation are described in “Liposome Technology” (1984. Gregoriadis G. ed. CRC Press Inc Boca Raton Fla. Vol I II & III).

Methods for preparation of drug containing liposomes are also known to one skilled in the art. The liposomes may be prepared by a variety of techniques as described herein above. Generally, a therapeutic drug is incorporated into liposomes by adding the drug to the vesicle-forming lipids prior to liposome formation, to entrap the drug in the formed liposome. If the drug is hydrophobic the drug is added directly to the hydrophobic mixture. If the drug is hydrophilic the drug can be added to the aqueous medium that covers the thin film of evaporated lipids.

U.S. Pat. No. 4,235,871 to Papahadjopoulos et al. describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture that, upon agitation or dispersion in an aqueous media, is converted to large ULVs. U.S. Pat. No. 4,016,100 to Suzuki et al. describes another method of encapsulating agents in unilamellar vesicles by freezing/thawing an aqueous phospholipid dispersion of the agent and the lipids. Other traits may be added to drug encapsulating liposomes to increase their therapeutic efficiency. For example, U.S. Pat. No. 5,527,528 to Allen et al. discloses liposomes containing an anti-tumor compound further comprising a surface coating of polyethylene glycol chains, at a surface concentration sufficient to extend the blood circulation time of the liposomes several fold over that of liposomes in the absence of such coating, and surface-attached antibody molecules effective to bind specifically to tumor-associated antigens present at the tumor site. U.S. Pat. No. 6,043,094 to Martin et al. also describes liposomes with outer surfaces that contain an affinity moiety effective to bind specifically to a target surface at which the therapy is aimed. This patent also discloses the use of a hydrophilic polymer coating effective in shielding the affinity moiety from interaction with the target surface. The hydrophilic polymer coating is made up of polymer chains which are covalently linked to surface lipid components in the liposomes through releasable linkages. The administered liposomes are allowed to circulate systemically until a desired bio-distribution of the liposomes is achieved, and a releasing agent is then administered to the subject in an amount effective to cause release of a substantial portion of the releasable linkages in the administered liposomes, exposing the affinity agent to the target surface. US Patent application 2001/0051183 to Martin et al. discloses the use of such liposomes for localizing an anti-tumor agent, for example anthracycline, to a solid tumor via the blood stream.

International Patent Application No. WO 02/076427 discloses liposome that employs phosphatidyl ethanolamine, cholesterol hemisuccunate and cholesterol in a ratio of 7:4:2 for administration of therapeutic agent to a macrophage. The liposomes are stable at physiological pHs, while at the same time being fusogenic at acidic pHs. This property allows for the delivery of the therapeutic agent into the cytosol, and subsequently the nucleus, of the macrophage. The liposome composition is useful in the treatment of macrophage associated diseases or conditions.

U.S. Pat. No. 5,605,703 to Lambiez et al. discloses the use of anti-free radicals agents within liposomes encapsulating anti-neoplastic agents, specifically doxorubicin, to reduce the toxicity of the encapsulated drug.

A method for the production of emulsomes, fusogenic vesicle having characteristics of both liposomes and nanoemulsions is described for example in U.S. Pat. No. 5,576,016 to Amselem et al.

According to one embodiment the liposomes of the present invention comprise at least one of the following substances: phospholipids of natural or synthetic origin; phospholipids combined with glycerides; phospholipids combined with polyethylene glycol (PEG); phosphoaminolipids; cerebroglucosides and gangliosides; optionally further comprising natural or synthetic cholesterol.

The existence of a hydrophobic compartment in both liposomes and the Haptides of the present invention allows the association of these compounds in the disclosed haptotactic-peptide liposomal compositions. Without wishing to be bound to any specific mechanism one possible advantage of said compositions is the non-specific interaction between the haptotactic peptides and the liposomes as well as the compounds contained within the liposome.

The liposomal composition of the present invention may further comprise biologically active compounds that are not readily taken up by a living cell due to a low permeability through the cell membrane, including, but not limited to polynucleotides, proteins, peptides, polysaccharides, hormones, drugs, vitamins, steroids, fluorescent dyes, radioactive markers and the like. These biologically active compounds may have a diagnostic, therapeutic or cosmetic activity.

According to another aspect, the present invention provides a method for enhancing liposomes uptake into cells, in vitro or in vivo.

According to one embodiment the present invention provides a method for enhancing liposomes uptake into cells comprising:

providing a haptotactic-peptide liposomal composition; and

contacting cells with the haptotactic-peptide liposomal composition;

wherein liposomal uptake by the cells is enhanced at least two fold compared to the uptake of the same liposomes absent the haptotactic peptide.

According to one embodiment, the provided Haptotactic Peptide-liposome composition is produced ab initio with a selected Haptide. According to another embodiment, the composition is produced extemporaneously using preformed vesicles combined with at least one type of Haptide.

According to one embodiment of the present invention the method of producing a haptotactic-peptide liposomal composition comprises the step of dispersing the liposomal components with a solution of at least one type of haptotactic peptide in an aqueous buffer.

In addition to the ability to elicit haptotactic response, the haptotactic peptides of the present invention are readily taken up and internalized by cells of various types, as exemplified herein below. Both phenomena are utilized for facilitating the uptake of liposomes into the cytoplasmic compartment of a cell, as schematically illustrated herein below. Dispersing or mixing lipophilic components and amphiphilic components of the liposomes with a selected Haptide in an aqueous solution results in the production of haptotactic-peptide liposomal composition. The composition achieves the cell binding and cell internalization properties of the haptotactic peptide, and it can readily attach to and be taken up by the target cells or tissue.

According to one embodiment, the cells to which the liposomes are directed are selected from a group consisting of mammalian cells including leukocytes, and cells from mesenchymal origin including astrocytes, chondrocytes, dendritic cells, endothelial cells, fibroblasts, glial cells, neurons, kidney cells, liver cells, melanocytes, mesenchymal cells, myofibroblasts, monocytes, parenchymal cells, pancreatic cells, smooth muscle cells and thyroid cells as well as malignant and transformed cells of any origin.

According to yet another aspect, the present invention provides a method for using haptotactic-peptide liposomal compositions for enhancing the intracellular uptake of biologically active molecules that would otherwise have low-permeability through the cell membrane.

According to one embodiment the present invention provides a method for using haptotactic-peptide liposomal compositions for enhanced intracellular uptake of biologically active molecules characterized by low-permeability through the cell membrane, the method comprising:

providing a haptotactic-peptide liposomal composition wherein the liposomes further comprise biologically active molecules characterized by low-permeability through the cell membrane; and

contacting cells with the haptotactic-peptide liposomal composition;

wherein the molecules uptake is enhanced at least two fold compared to the uptake of molecules detached from said haptotactic-peptide liposomal composition.

According to this aspect the haptotactic-peptide liposomal compositions serve as a vehicle for molecules having a low permeability through the cell membrane, such vehicle augmenting the uptake of such molecules into cells.

According to one embodiment, the haptotactic peptide-liposomal composition which further comprises molecules characterized by low-permeability through the cell membrane is produced ab initio with at least one type of Haptide and at least one type of the low-permeability molecule. According to another embodiment, the composition is produced extemporaneously using preformed vesicles comprising at least one type of low-permeability molecule combined with at least one type of Haptide.

The biologically active molecules may be hydrophilic or hydrophobic. Hydrophilic molecules are present within the hydrophilic compartments of the liposomes—the core or the volume between two lipid layers. Hydrophobic molecules are distributed within the hydrophobic compartments of the liposomes that form the lipid layers themselves.

According to one embodiment, the biologically active molecules are selected from the group consisting of polynucleotides, proteins, peptides, polysaccharides, hormones, drugs, vitamins, steroids, fluorescent dyes, radioactive markers and the like.

According to a further aspect the present invention provides pharmaceutical and cosmetic composition comprising haptotactic-peptide liposomal compositions wherein the liposomes further comprise an active ingredient having diagnostic, therapeutic or cosmetic activity, respectively.

According to one embodiment the present invention provides a pharmaceutical composition comprising haptotactic peptide-Liposomal composition, wherein the peptides are haptotactic peptides characterized in that they elicit cell attachment activity and are internalized by cells and the liposomes further comprise an active ingredient having a diagnostic or therapeutic activity, further comprising a pharmaceutically acceptable diluent or carrier.

According to one preferred embodiment, the active ingredient within the liposomes of the pharmaceutical composition is selected from the group consisting of a cytotoxic compound, a cytostatic compound, an antisense compound, an anti-viral agent, a specific antibody and an imaging agent.

According to another embodiment the present invention provides a cosmetic composition comprising haptotactic peptide-liposomal composition, wherein the peptides are haptotactic peptides characterized in that they elicit cell attachment activity and are internalized by cells and the liposomes further comprise an active ingredient with a cosmetic beneficial effect, further comprising a cosmetically acceptable diluent or carrier.

According to yet another aspect the present invention relates methods for treating a subject in need thereof with the pharmaceutical or cosmetic compositions of the present invention.

According to one embodiment the present invention provides a method for enhancing the delivery of a pharmaceutical agent into cells comprising the step of administering to a subject in need thereof a therapeutically effective amount of haptotactic peptide-liposomal pharmaceutical composition wherein the liposomes of the composition further comprise the pharmaceutically effective agent.

According to another embodiment the present invention provides a method for enhancing the delivery of a diagnostic agent into cells comprising the step of administering to a subject in need thereof a diagnostically effective amount of haptotactic peptide-liposomal pharmaceutical composition, wherein the liposomes of the composition further comprise the diagnostically effective agent.

According to yet another embodiment the present invention provides a method for enhancing the delivery of a cosmetically effective liposomes into cells comprising the step of administering to a subject in need thereof a haptotactic peptide-liposomal cosmetic composition wherein the liposomes of the composition have a cosmetic beneficial effect. These liposomes may further comprise a cosmetically effective agent.

According to further embodiment the present invention provides the use of a haptotactic peptide-liposomal pharmaceutical composition wherein the liposomes of the composition further comprise a pharmaceutically effective agent for enhancing the delivery of the pharmaceutically effective agent into cells.

According to yet further embodiment the present invention provides the use of a haptotactic peptide-liposomal pharmaceutical composition wherein the liposomes of the composition further comprise a diagnostically effective agent for enhancing the delivery of the diagnostically effective agent into cells.

According to another embodiment the present invention provides the use of a haptotactic peptide-liposomal pharmaceutical composition wherein the liposomes of the composition have a cosmetically beneficial effect for enhancing the delivery of the cosmetically effective liposomes into cells. These liposomes may further comprise a cosmetically effective agent.

Pharmaceutical compositions of the present invention may be manufactured by processes well known in the art, e.g. by means of conventional mixing, dissolving, emulsifying, encapsulating, entrapping or lyophilizing processes.

Pharmaceutical composition for use in accordance with the present invention thus may be formulated in conventional manner using one or more acceptable diluents or carriers comprising excipients and auxiliaries, which facilitate processing of the active liposomes into preparations, which can be used pharmaceutically. Proper formulation is dependent on the route of administration chosen. More particularly, the present invention relates to pharmaceutical compositions for administering orally, parenterally, topically or by inhalation.

The haptotactic-peptide liposomal compositions of the present invention and the principle of using same may be better understood with reference to the following non-limiting examples.

### EXAMPLES

Chemicals and reagents

Clinical grade human fibrinogen and thrombin were purified from blood by New York Blood Center and Vitex Inc. (New York, N.Y.). Tissue culture media, serum, bovine serum albumin (BSA) and other reagents were purchased from standard commercial sources for laboratory supply, mainly from Biological Industries (Beit-HaEmek, Israel), Sigma Chemicals (Israel and St. Louis, Mo.) and GIBCO (Grand Island, N.Y., N.Y.); other reagents were from Sigma Chemicals (Israel and St. Louis, Mo.). Liposomes containing entrapped Rhodamine, composed of hydrogenated phosphatidyl serine, PEG and cholesterol, were provided by Professor A. Gabizon (Hadassah University Hospital, Dept. of Oncology). Clinical grade liposomes containing doxorubicin were commercially available for chemotherapy as the drug Doxil (Johnson & Johnson).

The 17-21 peptides sequences examined in the examples of the present invention (Table 1) were synthesized at a few different facilities, namely the Microchemistry facility of the New York Blood Center (New York, N.Y.) or by SynPep Corporation (Dublin, Calif.). Other batches of peptides were synthesized by the Inter-departmental Services of the Medical School at the Hebrew University (Jerusalem) or by Alpha Diagnostics International (San Antonio, Tex.).

TABLE 1
Synthesized peptides and their homology to Cβ

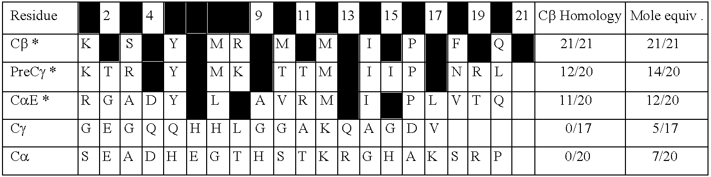

* Termed as Haptide
□ Homologous amino acid

Cell Cultures

The cell types used in the examples of the present invention were obtained and cultured as previously described (Gorodetsky R. et al. 1998. J Lab Clin Med 131: 269-280). Briefly, normal human skin fibroblasts (HF) were isolated from skin biopsies of young normal volunteers and cultured for no more than 14 passages. Normal bovine aortic endothelial cells (BAEC) were isolated from fresh thoracic aortas collected at slaughterhouse from sacrificed young animals and were kept in culture for up to 12-15 passages. The cell cultures were maintained at 37° C. in a water-jacketed CO₂ incubator, and were harvested by trypsin/EDTA solution with 1-2 passages per week in a split ratio of 1:10 for rapidly proliferating, transformed cells and 1:4 for normal cell types.

### Example 1

### Cell Binding Activity of Haptides and Fibrinogen Bound to Sepharose Beads, and Cellular Internalization of their Free Form

Cell Adhesion (Haptotaxis) Assay

The attachment of Sepharose beads (SB)-ligand to cells in nearly confluent cultures grown on plastic was measured as previously described (Gorodetsky R. et al. 1998. J Lab Clin Med 131, 269-280; Gorodetsky R. et al. 1999. J Invest Dermatol 112:866-872). Essentially, about 20-150 μl of suspended (50% v/v) SB-Haptide or SB-fibrinogen were added to near confluent cell cultures in 6-24 well plates and dispersed by gentle shaking for 1 min. The plates were then incubated for up to 4 days. After one day the number of SB tethered to cell layer was counted with an inverted phase or Numarsky microscopy. Typically, ˜300 SB (but not less then 200) were counted in each well, and the ratio of the number of SB attached to the cells in each well, was calculated relative to the total number of SB. At least 3 wells were measured for each variant and each experiment was repeated at least 3 times.

Monitoring Peptide Uptake by Cells with Confocal Laser Fluorescence Microscopy.

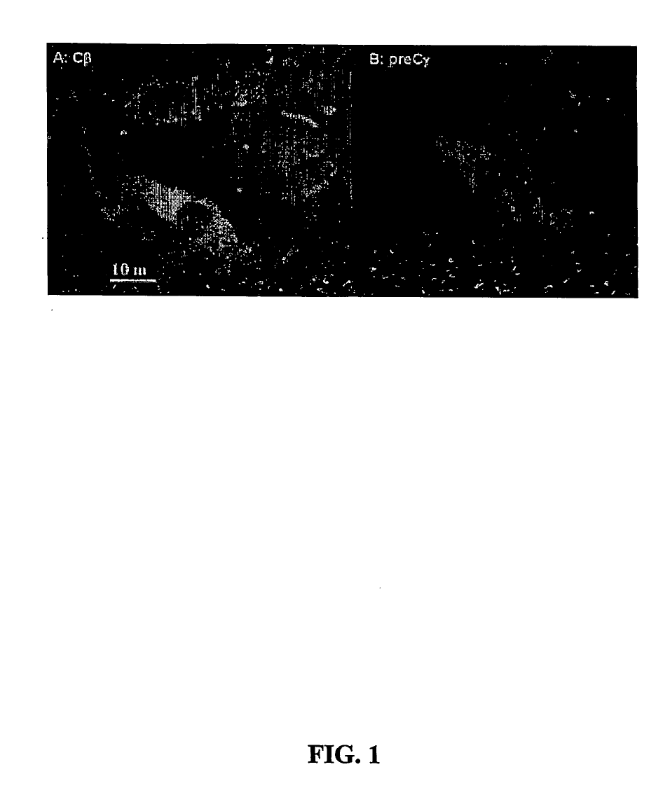

Fluorescent microscopy was carried out with Olympus or Nikon fluorescence microscopes. Confocal laser microscopy was done with a computerized Zeiss Confocal Axiomate microscope (LSM410) with multiple excitation wavelengths. Peptides were tagged with fluorescein isothiocyanate (FITC) for visualization. For examination of ^{FITC}peptides uptake by human fibroblasts (HF) the cells were grown on glass coverslips to near confluence and then incubated with 100 μg/ml (40 μM) ^{FITC}peptides at 37° C. After 1 h, the cells were washed and fixed in 0.5% buffered glutaraldehyde. Coverslips with the cells were placed on a microscope slide with PBS-glycerol 80% with 2% DABCO and examined. The representative fields of cells were visualized by Numarski optics and confocal scans of cell fluorescence intensity in 1 μm slices were recorded at the FITC wavelength (excitation 488 nm, emission 515 nm) and later reconstructed. Uptake of ^{FITC}ligand by HF was scored visually (0-4). The fluorescent haptotactic peptides were clearly seen taken up by the HF cells, and the nuclei remained relatively free of ^{FITC}Haptide (FIGS. 1A & B). Haptides were found to accumulate in nano-aggregate structures within the cytoplasm and eventually the fluorescence gradually faded. By contrast, the uptake of the control ^{FITC}Cα peptide by the cells was negligible.

TABLE 2
Haptotactic activity (%) and cell uptake of
fibrinogen (fib) and different peptides

| | % Haptotaxis | % Haptotaxis | Cell Uptake* |
|---|---|---|---|
| Ligand | (BAEC, 1 day) | (HF, 1 day) | (HF) |
| Fib | 100 | 100 | +2 |
| Cα | 0 | 0 | 0-+1 |
| CαE | 75 | 60 | +3 |
| Cβ | 100 | 100 | +4 |
| Cγ | 0 | 0 | nr** |

*Visual evaluation from fluorescence micrographs of cell with labeled Haptides
**nr—not recorded

Uptake and internalization of free Haptides (Table 2) seemed to be positively correlated with the haptotactic activity of peptides bound to SB-matrix towards the cells (Columns 2 & 3, Table 2).

### Example 2

### Binding of ^{FITC}Haptide or ^{FITC} Fibrinogen to Liposomes

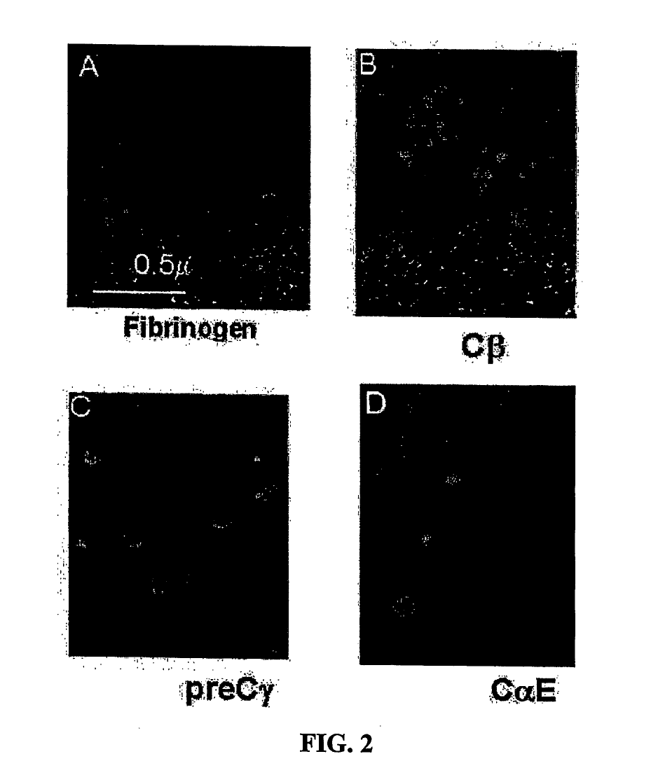

Liposomes (100 μL) composed of hydrogenated phosphatidyl serine, PEG and cholesterol (Sundar S. & Gregoriadis G. 2001. Lancet 357 (9258):801-2; U.S. Pat. No. 6,083,530), were suspended in Tris saline, pH 7.2. Ten μg/ml ^{FITC}-ligand, i.e. Cβ, preCγ, CαE or fibrinogen, were then added. The mixture was placed on a glass slide and examined by confocal laser microscopy using a computerized Zeiss Confocal Axiomate microscope (LSM410) with multiple excitation wavelengths. Digital images were stored in the computer for further image reconstruction. As shown in FIG. 2, ^{FITC}fibrinogen (A), ^{FITC}Cβ (B), ^{FITC}preCγ (C) and ^{FITC}CαE (D), bound to the liposomes (initial size ˜100 nm) and induced their aggregation into larger particles. These images indicate that the peptides as well as fibrinogen were hydrophobic, to the extent that they preferentially distributed from the aqueous solution to the liposomes lipid bilayer.

### Example 3

### The Haptotactic Peptide-Liposomal Composition

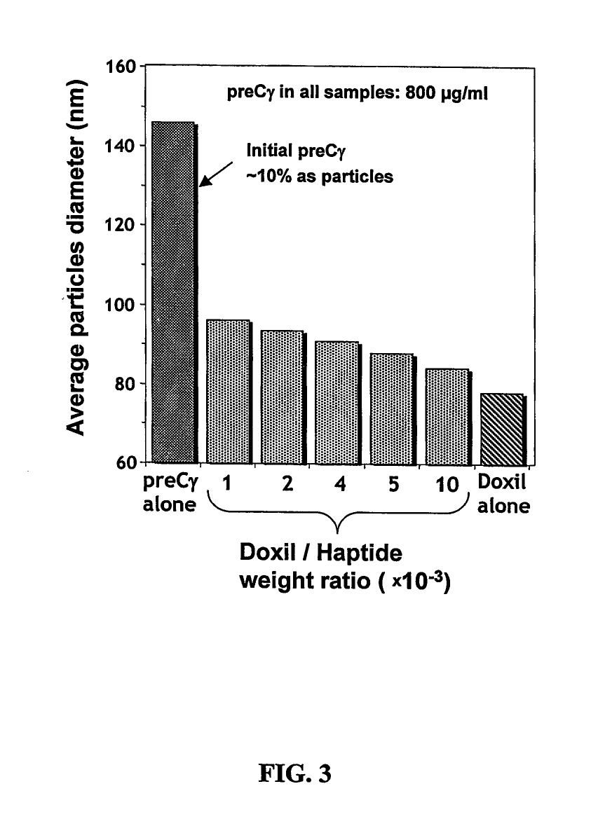

It has been previously observed by gel filtration that haptides tend to form aggregates (Gorodetsky R, Vexler A, Shamir M, An J, Levdansky L, Shimeliovich I, Marx G. Exp Cell Res. 287:116-129 (2003). A solution of the Haptide preCγ (1 mg/mL) was passed through a 0.45 μm filter and then measured by dynamic light scattering at 25° C. The average particle size (a mean value of three measurements) was ˜145 nm (FIG. 3), showing that such particle is an aggregate of few hundreds of preCγ monomers.

Similarly, Doxil liposomes were measured and observed to have a mean diameter of 80 nm. However, when the preCγ-liposomal composition was formed, the preCγ Haptides aggregates disappeared in correlation with the liposome/Haptide weight ratio (FIG. 3). These results demonstrate that the solubilized Haptides dissolve within the liposome lipid bilayer. At a doxil/haptide weight ratio of >10⁻², the almost totally all the Haptides aggregates were solubilized within the liposomes as indicated by the disappearance of larger Haptide particles, forming the haptotactic-peptide liposomal composition.

### Example 4

### Uptake of Haptotactic Peptide-Liposomal compositions by HF and BAEC Cells

Human fibroblasts or bovine aortic endothelial cells were seeded and grown in CO₂ incubator for 24 hours in a 4-chamber cell culture coverslip slide (source) used for microscopy. Then, they were exposed to either free ^{FITC}Haptide or to a ^{FITC}Haptotactic Peptide-Liposomal composition. Two liposome types were examined, namely liposomes composed of hydrogenated phosphatidyl serine, PEG and cholesterol, containing the fluorescent dye Rhodamine; and clinical grade liposomes containing the anti-cancer drug doxorubicin (Doxil liposomes). Both rhodamine and doxorubicin are fluorescent compounds. Thus, the liposome penetration and distribution within cells could be tracked by fluorescence microscopy or FACS. Typically, 20 μl of fluorescent Haptotactic Peptide-Liposomal composition were mixed with 180 μl PBS and added to the cell chamber. Incubation was continued for up to 1 hour at 37 or 4° C. The incubation was stopped at different time points by two subsequent washes with PBS, followed by fixation with 500 μl of 4% formaldehyde for 1 hr. The samples were then washed again with PBS. Coverslips with the fixed cells were covered by PBS-glycerol 80%, DABCO 2%, and an additional coverslip for microscopic examination.

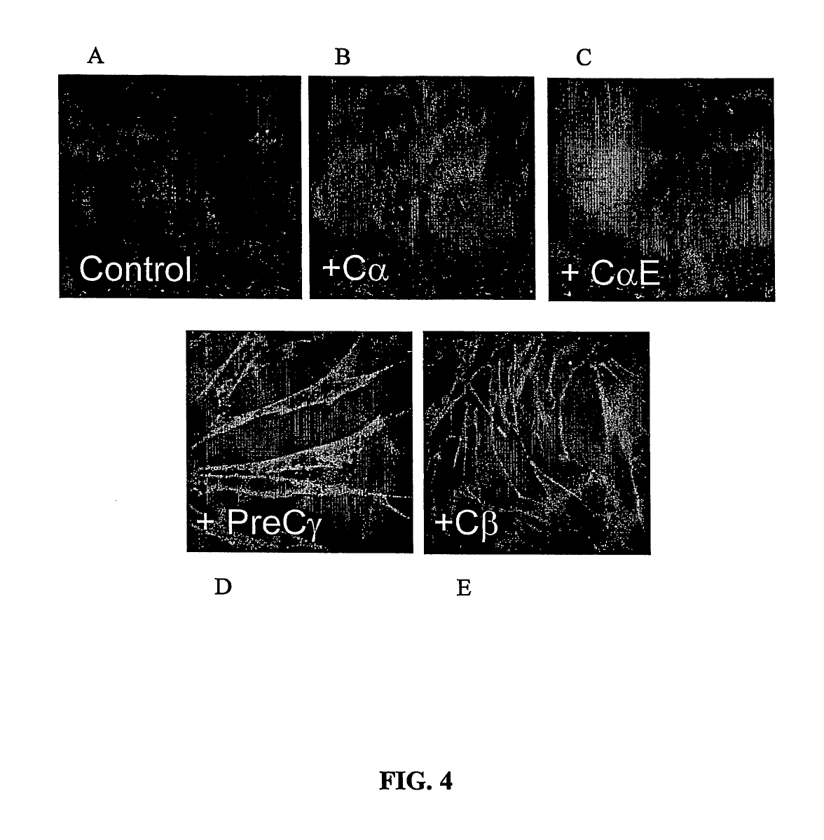

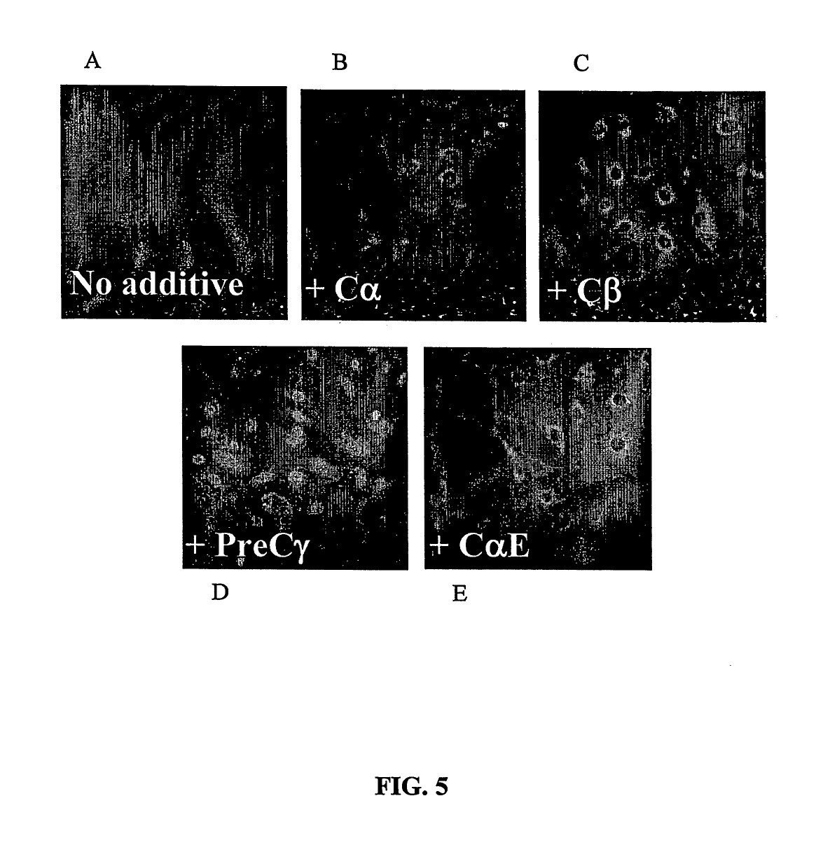

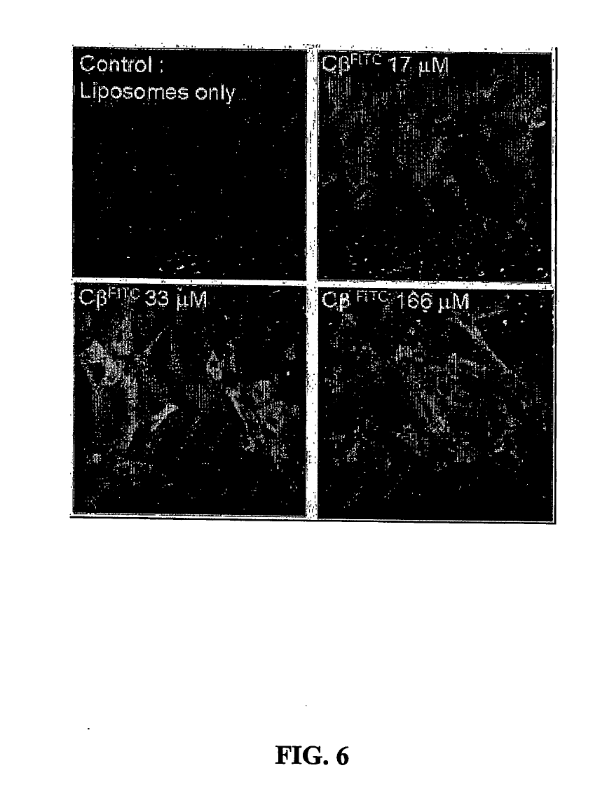

Haptotactic Peptide-Liposomal composition comprising the Haptides Cβ or preCγ facilitated liposome uptake into the cytoplasm of fibroblasts (FIGS. 4D & E) as well as of endothelial cells (FIGS. 5C&D). As expected, Cα did not induce such an uptake, concomitant with its lack of haptotactic activity (FIGS. 4B and 5B). The uptake of free liposomes into the cells under the examined conditions was also much lower (FIGS. 4A and 5A). Low induction was also observed for liposome uptake mediated by CαE into fibroblasts cell (FIG. 4C). However, this Haptide was efficient in enhancing doxorubicin-liposome (Doxil) uptake into endothelial cells (FIG. 5E). FIG. 6 shows concentration-dependent liposomes uptake mediated by Haptide Cβ. It can be clearly seen that the haptotactic peptide Cβ increased liposome uptake by human fibroblasts.

Uptake of Haptides-Doxil liposomes compared to Doxil liposomes by human fibroblast was also examined by measuring doxorubicin effect on cell survival.

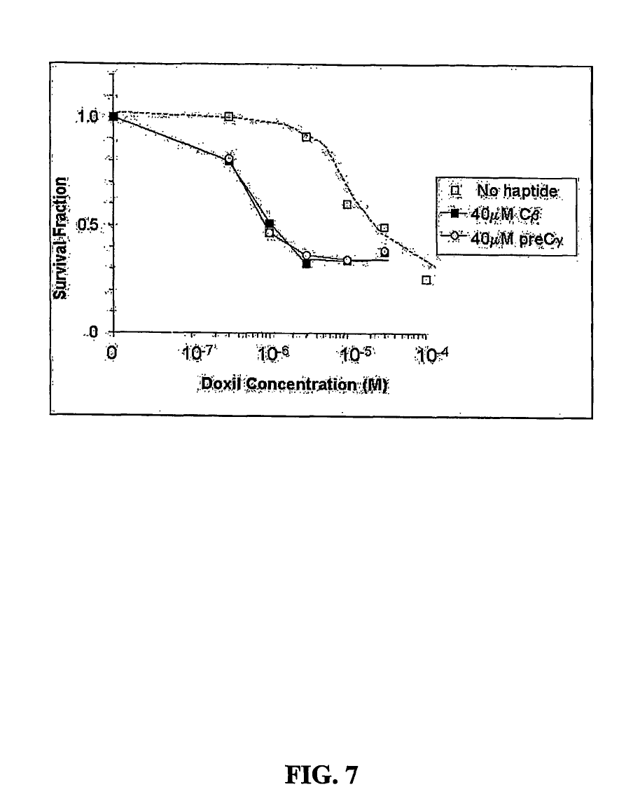

Normal human fibroblasts (HF) were plated on multi-well plate (˜2,000 cells/well) in normal fibroblasts cell culture medium. The attached cells were then incubated with different concentrations of Doxil or Haptide-Doxil (Cβ or preCγ) for 2 h. The haptotactic peptides of the present invention alone have no effect on cell survival. As shown in FIG. 7 Doxil alone reduced survival to ˜50% at concentrations of about 4×10⁻⁵ M while with both Haptides a concentration of only 10⁻⁶ was required to reach the same activity. These results demonstrate that the examined Haptides can significantly enhance the uptake of Doxil liposomes as reflected by the elevated cytotoxicity.

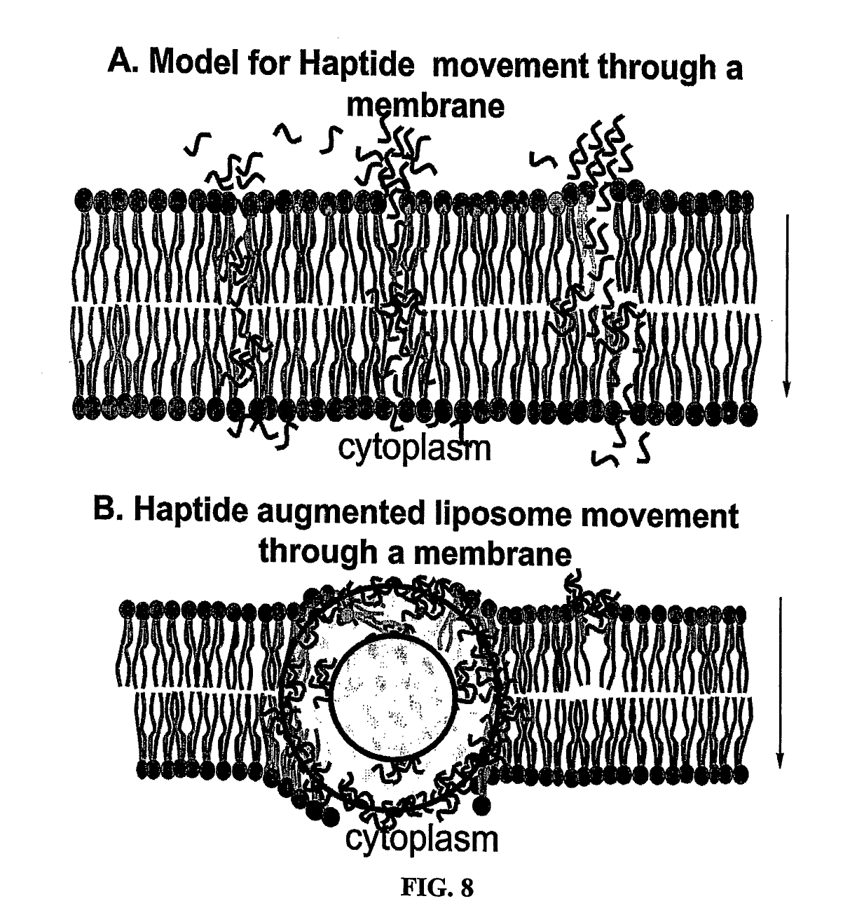

The principle underlying liposome uptake mediated by haptotactic peptides is schematically illustrated in FIG. 8: By their nature, Haptides are attached to the cell membrane, and as shown in the present invention are readily taken through the membrane into the cell (FIG. 8A). Haptotactic-peptide liposomal composition is generated due to the hydrophobic compartments in both substances. The composition maintains the parent Haptide properties, enhancing liposome uptake by the cells.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. The means, materials, and steps for carrying out various disclosed chemical structures and functions may take a variety of alternative forms without departing from the invention. Thus the expressions “means to . . . ” and “means for . . . ”, or any method step language, as may be found in the specification above and/or in the claims below, followed by a functional statement, are intended to define and cover whatever chemical structure, or whatever function, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

1. A haptotactic-peptide liposomal composition comprising at least one type of peptide and one type of liposome, wherein the peptide is characterized in that it elicits cell attachment responses and having an amino acid sequence that is at least 60% homologous to a haptotactic peptide present within the carboxy termini of fibrinogen chains, and the liposome has at least one lipid bilayer enclosing an aqueous compartment.

2. The composition of claim 1, wherein the peptide sequence is at least 80% homologous to a haptotactic peptide present within the carboxy termini of fibrinogen chains.

3. The composition of claim 1, wherein the peptide is selected from the group consisting of SEQ ID NOs. 1-3 and analogues, derivatives, homologues fragments or mimetics thereof, providing they retain cell attachment activity.

4. The composition of claim 1, wherein the peptide is selected from the group consisting of SEQ ID NOs. 4-7 and analogues, derivatives, homologues, fragments or mimetics thereof, providing they retain cell attachment activity.

5. The composition of claim 1, wherein the peptide is selected from the group consisting of SEQ ID NOs. 8-12 and analogues, derivatives, homologues, fragments or mimetics thereof, providing they retain cell attachment activity.

6. The composition of claim 1, wherein the peptide is selected from the group consisting of SEQ ID NO: 13 and SEQ ID NO: 14 and analogues, derivatives, homologues, fragments or mimetics thereof, providing they retain cell attachment activity.

7. The composition of claim 1 characterized in that uptake of the haptotactic-peptide liposomal composition by mammalian endothelial or fibroblast cells is enhanced at least 2 fold compared to the uptake of said liposomes absent said haptotactic peptide.

8. The composition of claim 1 wherein the liposomes comprise at least one of the group consisting of phospholipids of natural or synthetic origin; phospholipids combined with polyethylene glycol; phospholipids combined with glycerides; phospho amino lipids cerebroglucosides and gangliosides; optionally further comprising natural or synthetic cholesterol.

9. The composition of claim 1 wherein the liposomes further comprise biologically active compound.

10. The composition of claim 9 wherein the biologically active compound is selected from the group consisting of polynucleotides, proteins, peptides, polysaccharides, hormones, drugs, steroids, fluorescent dyes and radioactive markers.

11. A method for enhancing liposome uptake into cells, comprising providing a haptotactic-peptide liposomal composition according to claim 1, and contacting cells with said composition, wherein liposomal uptake by the cells is enhanced at least two fold compared to the uptake of said liposomes absent the haptotactic peptide.

12. The method of claim 11 wherein the haptotactic-peptide liposomal composition is produced ab initio with at least one type of Haptide.

13. The method of claim 11 wherein the haptotactic-peptide liposomal composition is produced extemporaneously using preformed vesicles combined with at least one type of Haptide.

14. The method of claim 12 wherein the method of producing the haptotactic-peptide liposomal composition comprises the step of dispersing lipophilic and amphiphilic components and at least one type of haptotactic peptide in an aqueous solution.

15. The method of claim 11 wherein the peptide sequence is at least 80% homologous to a haptotactic peptide present within the carboxy termini of fibrinogen chains.

16. The method of claim 11 wherein haptotactic peptide is selected from the group consisting of SEQ ID NOs. 1-3 and analogues, derivatives, homologues, mimetics or fragments thereof, providing they retain cell attachment activity.

17. The method of of claim 11 wherein haptotactic peptide is selected from the group consisting of SEQ ID NOs. 4-7 and analogues, derivatives, homologues, mimetics or fragments thereof, providing they retain cell attachment activity.

18. The method of claim 11 wherein haptotactic peptide is selected from the group consisting of SEQ ID NOs. 8-12 and analogues, derivatives, homologues, mimetics or fragments thereof, providing they retain cell attachment activity.

19. The method of claim 11 wherein haptotactic peptide is selected from the group consisting of SEQ ID NO: 13 and SEQ ID NO:14 and analogues, derivatives, homologues, mimetics or fragments thereof, providing they retain cell attachment activity.

20. The method of claim 11 wherein the lipid phase of the liposomes comprise at least one of the group consisting of phospholipids of natural or synthetic origin; phospholipids combined with polyethylene glycol; phospholipids combined with glycerides; phosphoaminolipids cerebroglucosides and gangliosides; optionally further comprising natural or synthetic cholesterol.

21. The method of claim 11 wherein the cells are selected from the group consisting of mammalian cells including leukocytes, and cells from mesenchymal origin including astrocytes, chondrocytes, dendritic cells, endothelial cells, fibroblasts, glial cells, neurons, kidney cells, liver cells, melanocytes, mesenchymal cells, myofibroblasts, monocytes, parenchymal cells, pancreatic cells, smooth muscle cells and thyroid cells, malignant and transformed cells.

22. A method for enhancing intracellular uptake of biologically active compounds characterized by low-permeability through the cell membrane using a haptotactic-peptide liposomal composition, the method comprising the steps of providing a haptotactic-peptide liposomal composition, wherein the liposomes comprise biologically active molecules characterized by low permeability through cell membrane, and contacting cells with the haptotactic-peptide liposomal composition, wherein the molecules uptake is enhanced at least two fold compared to the uptake of said molecules detached from said haptotactic-peptide liposomal composition.

23. The method of claim 22 wherein the haptotactic-peptide liposomal composition is produced ab initio with at least one type of Haptide and at least one type of biologically active molecule.

24. The method of claim 22 wherein the haptotactic-peptide liposomal composition is produced extemporaneously using preformed vesicles comprising at least one type of biologically active molecule combined with at least one type of Haptide.

25. The method of claim 23 wherein the method of producing the haptotactic-peptide liposomal composition comprises the step of dispersing lipophilic and amphiphilic components, at least one type of hap to tactic peptide and at least one type of biologically active molecule in an aqueous solution.

26. The method of claim 22 wherein the peptide sequence is at least 80% homologous to a haptotactic peptide present within the carboxy termini of fibrinogen chains.

27. The method of claim 22 wherein haptotactic peptide is selected from the group consisting of SEQ ID NOs. 1-3 and analogues, derivatives, homologues, mimetics or fragments thereof, providing they retain cell attachment activity.

28. The method of claim 22 wherein haptotactic peptide is selected from the group consisting of SEQ ID NOs. 4-7 and analogues, derivatives, homologues, mimetics or fragments thereof, providing they retain cell attachment activity.

29. The method of claim 22 wherein haptotactic peptide is selected from the group consisting of SEQ ID NOs. 8-12 and analogues, derivatives, homologues or fragments thereof, providing they retain cell attachment activity.

30. The method of claim 22 wherein haptotactic peptide is selected from the group consisting of SEQ ID NO:13 and SEQ ID NO: 14 and analogues, derivatives, homologues or fragments thereof, providing they retain cell attachment activity.

31. The method of claim 22 wherein the lipid phase of the liposomes comprise at least one of the group consisting of phospholipids of natural or synthetic origin; phospholipids combined with polyethylene glycol; phospholipids combined with glycosides; phosphoaminolipids cerebroglucosides and gangliosides; optionally further comprising natural or synthetic cholesterol.

32. The method of claim 22 wherein the cells are selected from a group consisting of mammalian cells including leukocytes, and cells from mesenchymal origin including astrocytes, chondrocytes, dendritic cells, endothelial cells, fibroblasts, glial cells, neurons, kidney cells, liver cells, melanocytes, mesenchymal cells, myofibroblasts, monocytes, parenchymal cells, pancreatic cells, smooth muscle cells, thyroid cells, malignant and transformed cells.

33. The method of claim 22 wherein the biologically active compound within the liposomes is selected form the group consisting of polynucleotides, proteins, peptides, polysaccharides, hormones, drugs, steroids, fluorescent markers and radioactive markers.

34. A pharmaceutical composition comprising Haptotactic Peptide-Liposomal composition, wherein the liposomes comprise at least one active ingredient having a diagnostic or therapeutic activity, said liposomes are formulated in a pharmaceutically acceptable diluent or carrier.

35. The pharmaceutical composition of claim 34 wherein the active ingredient is selected from the group consisting of a cytotoxic compound, a cytostatic compound, an antisense compound, an anti-viral agent, a specific antibody and an imaging agent.

36. A cosmetic composition comprising Haptotactic Peptide-Liposomal composition, wherein the liposomes have a cosmetic beneficial effect.

37. A method for enhancing the delivery of a pharmaceutical agent into cells comprising the step of administering to a subject in need thereof a therapeutically effective amount of a haptotactic peptide-liposomal pharmaceutical composition wherein the liposomes of the composition further comprise a pharmaceutically effective agent.

38. The method of claim 37 wherein the pharmaceutical composition is administered parenterally, topically, orally or by inhalation.

39. A method for enhancing the delivery of a diagnostic agent into cells comprising the step of administering to a subject in need thereof a diagnostically effective amount of a haptotactic peptide-liposomal pharmaceutical composition wherein the liposomes of the composition further comprise a diagnostically effective agent.

40. The method of claims 39 wherein the pharmaceutical composition is administered parenterally, topically or orally.

41. A method for enhancing the delivery of cosmetically effective liposomes into cells comprising the step of administering to a subject in need thereof a haptotactic peptide-liposomal composition wherein the liposomes of the composition have a cosmetic beneficial effect.

42. The method of claim 41 wherein the liposomes further comprise an active ingredient having a cosmetically beneficial effect.

43. The method of claims 41 wherein the cosmetic composition is administered topically.

44. (canceled)

45. (canceled)

46. (canceled)

47. (canceled)


SEQUENCE LISTING (14 sequences)

SEQ ID NO: 1 - protein, 21 aa, Artificial Sequence
  Equivalent to the c-terminus of fibrinogen beta-chain
KGSWYSMRKMSMKIRPFFPQQ

SEQ ID NO: 2 - protein, 21 aa, Artificial sequence
  Equivalent to the c-terminus of fibrinogen alphaE chain
RGADYSLRAVRMKIRPLTVTQ

SEQ ID NO: 3 - protein, 20 aa, Artificial sequence
  Equivalent to the c-terminus of fibrinogen pre-gama chain
KTRWYSMKKTTMKIIPFNRL

SEQ ID NO: 4 - protein, 19 aa, Artificial sequence
  Equivalent to the c-terminus of angiopoietin-1
KGPSYSLRSTTMMIRPLDF

SEQ ID NO: 5 - protein, 19 aa, Artificial sequence
  Equivalent to the c-terminus of angiopoietin-2
KGSGYSLKATTMMIRPADF

SEQ ID NO: 6 - protein, 19 aa, Artificial sequence
  Equivalent to the c-terminus of Tenascin X
KGFEFSVPFTEMKLRPNFR

SEQ ID NO: 7 - protein, 17 aa, Artificial sequence
  Equivalent to c-terminus of Microfibril associated protein
KGFYYSLKRPEMKIRRA

SEQ ID NO: 8 - protein, 8 aa, Artificial sequence
  Peptide equivalent to the c-terminus of fibrinogen beta-chain
KGSWYSMR

SEQ ID NO: 9 - protein, 10 aa, Artificial sequence
  Peptide equivalent to the c-terminus of fibrinogen beta-chain
KGSWYSMRKM

SEQ ID NO: 10 - protein, 10 aa, Artificial sequence
  Equivalent to the c-terminus of fibrinogen pre-gama chain
KTRWYSMKKT

SEQ ID NO: 11 - protein, 8 aa, Artificial sequence
  Equivalent to the c-terminus of angiopoietin-1
KGPSYSLR

SEQ ID NO: 12 - protein, 10 aa, Artificial sequence
  Equivalent to c-terminus of Microfibril associated protein
KGFYYSLKRP

SEQ ID NO: 13 - protein, 16 aa, Artificial sequence
  Consensus sequence
  MISC_FEATURE (3)..(4): Xaa is any  non-charged amino acid or is absent
  MISC_FEATURE (10)..(11): Xaa is any  non-charged amino acid or is absent
KGXXYSMRKXXMKIRP

SEQ ID NO: 14 - protein, 9 aa, Artificial sequence
  Consensus sequence
  MISC_FEATURE (3)..(4): Xaa is any non-charged amino acid or is absent
KGXXYSMRK